(19) United States
(12) Patent Application Publication
(10) Pub. No.: US 2026/0062669 A1
(43) Pub. Date: Mar. 5, 2026

(54) COMPOSITION COMPRISING AMNIOTIC FLUID STEM CELLS OR DERIVATIVES THEREOF AND USE THEREOF

(71) Applicant: U-NEURON BIOMEDICAL INC., New Taipei City (TW)

(72) Inventors: Shiaw-Min Hwang, New Taipei City (TW); Tzu-Hao Huang, New Taipei City (TW); Yu-Shan Huang, New Taipei City (TW); Fen-Ren Chien, New Taipei City (TW)

(73) Assignee: U-NEURON BIOMEDICAL INC., New Taipei City (TW)

(21) Appl. No.: 19/106,218

(22) PCT Filed: Aug. 29, 2023
(86) PCT No.: PCT/CN2023/115593

§ 371 Date: Feb. 25, 2025

Related U.S. Application Data
(60) Provisional application No. 63/373,964, filed on Aug. 30, 2022.

(51) Int. Cl. C12N 5/073; A61K 8/98; A61Q 7/00; A61Q 19/08

(52) U.S. Cl. CPC C12N 5/0605; A61K 8/982; A61Q 7/00; A61Q 19/08

(57) ABSTRACT

Provided is a composition including stem cell is derived from amniotic fluid of an artiodactyl animal. Also provided relates to uses of the composition for skin rejuvenation, skin treatment. promoting hair growth, and preventing hair loss, including administering an effective amount of the composition to the subject in need thereof.

### CROSS REFERENCE TO RELATED APPLICATION

This application claims the benefit of U.S. Provisional Application No. 63/373,964, filed on Aug. 30, 2022. The content of the application is incorporated herein by reference.

### BACKGROUND OF THE INVENTION

### 1. Field of the Invention

The present disclosure relates to a composition comprising amniotic fluid stem cells or derivatives thereof, particularly to a composition comprising amniotic fluid stem cells derived from artiodactyl animal stem cells or derivatives thereof.

### 2. Description of the Prior Art

Skin is the first barrier in animals and human against outside aggressions and it carries out important physical and chemical defenses. Moreover, skin is also the first line of immune defenses.

Skin is composed of three primary layers: the epidermis, dermis, and hypodermis. Each of these layers contains specific cells and it is well-known that the epidermis of skin sloughs millions of cells daily and creating demand for cell replacements as aging. There are also progenitors and stem cells proliferate and differentiate as part of natural repair and regeneration mechanism in the body. However, as aging, the proliferation of the cells may not able to keep up with the natural repair and this is when wrinkles may naturally become apparent. For example, creases, folds, or ridges in the skin such as crow's feet around corners of eyes and nasolabial folds on the face become apparent as aging. It has been shown that losing collagen is one of the major causes of wrinkle.

In addition, skin cells may become damaged by other physical impact means such as lacerations, abrasion injuries, scalds, diabetic ulcers, and bedsores caused by long-term bed rest. Wound healing is a complex and slow process in which new epithelial tissue and connective tissue are formed by the migration and proliferation during treatment.

The skin of an adult human is essentially covered with hair follicles, with approximately 100,000-150,000 covering the scalp. Hair follicles are believed to produce approximately 20 individual hair shafts over the life of the follicle as the follicle progresses through cycles of hair production, shedding, involution, and new growth.

Hair has many important physiological functions such as protection against external factors (including UV radiation), sebum, apocrine sweat and pheromones production, and thermoregulation. On an adult human scalp, at any particular time approximately 80-90% are in anagen; 10-20% in telogen, and 1-2% in catagen. Hair loss is prominent of the elderly population. It has been shown that hair follicle stem cells (HFSCs) are important for stimulate hair growth/hair promotion. Quiescence and activation of HFSCs can be trigger by both intrinsic and extrinsic signals. Imbalanced stem cell differentiation and altered stem cell activity are identified as important factors during hair loss. Thus, treatment that can promote HFSCs activation has been intensely studied.

The use of human stem cells widely described in prior art entails many problems which are evidence of the strong need to conduct further research in this area. Some of the main obstacles entailed by the use of embryonic cells are ethical questions, the risk of occurrence of genetic defects as well as risks of transferring viral and oncogenic diseases.

Thus, there is an unmet need in the art for compositions from stem cell lines and/or their derivatives would eliminate the risk of above obstacles, and methods of preparing skin care, hair promotion and cosmetic products from stem cells and or their derivatives obtained from a reliable source.

### SUMMARY OF THE INVENTION

The present disclosure provides a composition comprising a stem cell or a derivative thereof and cosmetically or pharmaceutically acceptable carrier, and the stem cell is derived from amniotic fluid of an artiodactyl animal.

In at least one embodiment, the artiodactyl animal includes, but not limited to, a pig, a peccary, a hippopotamus, an antelope, a deer, a giraffe, a camel, a llama, an alpaca, a sheep, a goat, and a cattle. In some embodiments, the artiodactyl animal is a deer.

In at least one embodiment, the derivative of the stem cell may be, but not limited to, a secretome, a conditioned medium, an exosome, an extracellular-vehicle, a secreted substance, or any combination thereof. In some embodiments, the derivative is a conditioned medium of the stem cell.

In at least one embodiment, the stem cell is negative for CD31, CD34, CD45, or any combination thereof. In some embodiments, the stem cell is derived from the amniotic fluid of the deer and negative for CD31, CD34, and CD45.

In at least one embodiment, the stem cell is positive for CD9, CD29, CD44, CD73, CD90, CD105, Nestin, Sox2, NANOG, or any combination thereof. In some embodiments, the stem cell is derived from the amniotic fluid of the deer and positive for CD9, CD29, CD44, CD73, CD90, CD105, Nestin, Sox2, and NANOG.

In at least one embodiment, the stem cell has an osteogenic differentiability, an adipogenic differentiability, a chondrogenic differentiability, or any combination thereof.

In at least one embodiment, the composition may be in a form selected from the group consisting of a gel, a lotion, an ointment, an emulsifier, a paste, a cream, a formulation to injection, a capsule, an oral liquid, a wafer, a film, a lozenge, a granule, a spray, and a pill.

In at least one embodiment, the stem cell or the derivative thereof is a sole active ingredient in the composition.

The present disclosure also provided a use of the composition for skin rejuvenation or skin treatment, comprising administering an effective amount of the composition of the present disclosure to a subject in need thereof. In at least one embodiment, the use may be a therapeutic use or a non-therapeutic use (e.g., a cosmetic use). Also provided is a method for skin rejuvenation or skin treatment, comprising administering an effective amount of the composition of the present disclosure to a subject in need thereof. The present disclosure additionally provides the composition for use in skin rejuvenation or skin treatment. Further provided is a use of the composition for manufacture of a medicament for skin rejuvenation or skin treatment.

In at least one embodiment, the composition of the present disclosure promotes synthesis of procollagen, collagen, or elastin in the subject.

In at least one embodiment, the skin rejuvenation comprises preventing, treating, or reducing an age-related feature on the skin of the subject. In some embodiments, the age-related feature may be, but not limited to, wrinkles, age spots, rough skin textures, dullness of skin, fine lines, visible pores, or loss of skin architecture due to decrease in collagen synthesis, elastin synthesis, or cell synthesis.

In at least one embodiment, the skin treatment comprises restoring damage of skin of the subject, reducing scar of damaged skin of the subject, enhancing restoration of the skin of the subject after a cosmetic or dermatological procedure, or preventing or reducing the wrinkle on the skin of the subject.

In some embodiments, the skin treatment comprises managing, treating, and/or preventing scarring, abnormal scars, abnormal wound healing, widened scar, hypertrophied scar, keloid, keloid scar, or wound-healing complication.

In further embodiments, the skin treatment comprises primary healing, wound closure, secondary healing, epithelialization, re-epithelialization, tertiary wound closure, or delayed primary closure.

In other embodiments, the compositions described herein are used to increase or decrease at the site of administration to a subject, inflammatory phase, proliferative phase, maturational phase, hemostasis, inflammation, collagen, clotting, thromboxane A2, prostaglandin 2a, hemorrhage, vasodilation, histamine, platelet, chemokine, epidermal growth factor, fibronectin, fibrinogen, clot formation, platelet degranulation, collagenase, fibroblast, collagen deposition, and insulin-like growth factor.

In at least one embodiment, the wrinkle is caused by age, obesity, injury, hormonal insufficiencies, drug side effects, or any combination thereof. In at least one embodiment, the wound is caused by cut, burn, acute or chronic trauma, surgical wound, or other chemical or physical impact.

The present disclosure also provided a use of the composition for promoting hair growth or preventing hair loss, comprising administering an effective amount of a composition of the present disclosure to a subject in need thereof. In at least one embodiment, the use may be a therapeutic use or a non-therapeutic use (e.g., a cosmetic use). Also provided is a method for promoting hair growth or preventing hair loss, comprising administering an effective amount of a composition of the present disclosure to a subject in need thereof. The present disclosure additionally provides the composition for use in promoting hair growth or preventing hair loss. Further provided is a use of the composition for manufacture of a medicament for promoting hair growth or preventing hair loss.

In at least one embodiment, the hair loss is caused by a disorder selected from the group consisting of androgenic alopecia, alopecia areta, and telogen effulvium.

In at least one embodiment, the composition promoting growth or migration of hair follicle dermal papilla cells in the subject.

In at least one embodiment, the composition is administered to the subject topically, subcutaneously, intradermally, or intramuscularly. In some embodiments, the administration may be via direct injection, condition medium, or topical administration to one or more areas of skin selected from the group consisting of scalp, face, neck, neckline, chest, back, arm, hand, leg, foot, or sole.

In at least one embodiment, the stem cell or the derivative thereof is a sole active ingredient in the composition of the present application.

In at least one embodiment, the composition of the present disclosure may be used alone or in combination with gauze, mask, ointment, tap, cosmetic composition, cosmetically acceptable vehicle or cosmetically acceptable carrier, a thickener, a filler, a moisturizer, an emulsifier, a humectant, a surfactant, a buffering or pH adjusting agent, a film forming agent, a preservative, an anti-oxidant, a fragrance, a solvent, a propellant, a colorant, or any combination thereof.

In at least one embodiment of the present disclosure, the stem cells of the artiodactyl animal are obtained by steps comprising:
(a) obtaining the amniotic fluid sample from the artiodactyl animal by amniocentesis;
(b) filtering the amniotic fluid with a sterile cell strainer;
(c) centrifuging and harvesting the stem cells from the amniotic fluid;
(d) culturing the stem cells in a culture medium;
(c) removing non-adherent cells and allowing adherent cells to grow as colonies; and when the adherent cells reach confluency,
(f) trypsinizing the adherent cells and passing the adherent cells for expansion or preservation.

In some embodiments of the present disclosure, the deer stem cells are obtained by steps comprising:
(a) obtaining deer amniotic fluid sample by amniocentesis, drawing amniotic fluid by syringe with a long needle under the ultrasound guidance, wherein the deer is more than 3 months of pregnancy;
(b) filtering fresh deer amniotic fluid with a sterile cell strainer and centrifuging the amniotic fluid sample at 200×g for a period of at least 5 minutes and removing supernatants;
(c) culturing the deer stem cells in a culture medium or a commercially available stem cell medium, wherein the culture medium is an alpha-modified minimum essential medium supplemented with about 10% to 20% fetal bovine serum and optionally with antibiotics;
(d) removing non-adherent cells by changing medium twice per week and allowing adherent cells to grow as colonies for at least 5 to 7 days and till reaching 80% to 90% confluency; and
(c) trypsinizing the adherent cells and passing the adherent cells at a seeding density of about 1000 to 9000 cells/cm² for expansion.

In some embodiments of the present disclosure, the basal medium can be MEM, DMEM, IMDM, RPMI 1640. In some embodiments of the present disclosure, the alpha-modified minimum essential medium is further supplemented with about 4 ng/ml basic fibroblast growth factor.

In some embodiments, the adherent cells are mammalian adherent cells. Any seeding density may be used which allows cells to form a confluent monolayer immediately or after a period of time in culture. In some embodiments, the seeding density may be about 10 cells/cm²-about 100,000 cells/cm², or about 100 cells/cm²-about 75,000 cells/cm², or about 500 cells/cm²-about 50,000 cells/cm², or about 500 cells/cm²-about 10,000 cells/cm², or about 500 cells/cm²-about 5,000 cells/cm², or about 500 cells/cm²-about 2,500 cells/cm², or about 1,000 cells/cm²-about 25,000 cells/cm², or about 2,000 cells/cm²-about 10,000 cells/cm², or about 3,000 cells/cm²-about 5000 cells/cm², but the present disclosure is not limited thereto. In some embodiments, the adherent cells may be stem cells, somatic cells, progenitor cells, mature cells, and/or cells from multiple germ layers, but the present disclosure is not limited thereto.

In some embodiments of the present disclosure, the essential medium may be supplemented with antibiotics, for example, penicillin, streptomycin, tetracyclines, and/or gentamicin, but the present disclosure is not limited thereto.

In some embodiments of the present disclosure, the essential medium may be supplemented with antifungal agents such as polyenes (amphotericin B), flucytosine, the imidazoles, triazoles (ketoconazole, fluconazole, itraconazole, and voriconazole), griseofulvin and ciclopirox, but the present disclosure is not limited thereto.

In at least one embodiment, the composition may further comprise glutamine, antioxidants and/or nicotinamide. In some embodiments, the antioxidant may be vitamin E, vitamin A, vitamin C, or any combination thereof.

In at least one embodiment, the stem cells derived from amniotic fluid bring about a better effect on promoting skin rejuvenation or skin treatment, enhancing hair growth, or preventing hair loss as compared with that derived from adult tissues such as adipose tissue or bone marrow. For instance, during the wound healing, the stem cells derived from the adult tissues may cause scare formation due to their promoting effect on myofibroblast differentiation and collagen synthesis. By contrast, the amniotic fluid stem cells or the derivatives thereof (e,g, a secretome, a conditioned medium, an exosome, an extracellular-vehicle, or a secreted substance) can achieve scar-free wound healing due to their anti-fibrotic properties through inhibition of myofibroblast differentiation and collagen synthesis. In some embodiment, the dAFSC shows more superior effect on promoting skin rejuvenation or skin treatment, enhancing hair growth, or preventing hair loss as compared with the human amniotic fluid-derived stem cells. For example, the dAFSC inhibited more myofibroblast differentation and collagen synthesis than other stem cells from other tissues of human.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will become more readily appreciated by reference to the following descriptions in conjunction with the accompanying drawings.

FIG. 1 shows fibroblast-like cell morphology originated from deer amniotic fluid under microscope (100×) in accordance with at least one embodiment of the present disclosure.

FIG. 2A and FIG. 2B show the expression of biomarkers CD9, CD29, CD31, CD34, CD44, CD45, CD73, CD90, CD105, Nestin, Sox2, GAPDII, and NANOG measured in the cells using the reverse transcription-polymerase chain reaction (RT-PCR) in accordance with at least one embodiment of the present disclosure.

FIG. 3A, FIG. 3B, FIG. 3C, and FIG. 3D show flow cytometry analysis of the antigens detected in the cells in accordance with at least one embodiment of the present disclosure. The quantification of FIG. 3A is shown in FIG. 3C, and the quantification of FIG. 3B is shown in FIG. 3D.

FIG. 4A and FIG. 4B show images of the cells (FIG. 4A) differentiating into osteocytes (FIG. 4B) in accordance with at least one embodiment of the present disclosure.

FIG. 4C and FIG. 4D show images of the cells (FIG. 4C) differentiating into adipocyte (FIG. 4D) in accordance with at least one embodiment of the present disclosure.

FIG. 4E and FIG. 4F respectively show 100× image (FIG. 4E) and 400× image (FIG. 4F) of the cells (FIG. 4C) differentiating into chondrocytes in accordance with at least one embodiment of the present disclosure.

FIG. 5 shows the cell proliferation (%) and procollagen synthesis rate (%) of the human skin fibroblasts treated with conditional medium of deer amniotic fluid stem cells (dASFC-CM) at different concentrations in accordance with at least one embodiment of the present disclosure. PC: positive control; NC: negative control.

FIG. 6 shows the wound healing area (%) of the human skin fibroblasts treated with dASFC-CM at different concentrations in accordance with at least one embodiment of the present disclosure. PC: positive control; NC: negative control.

FIG. 7 shows the cell viability (%) of the human skin fibroblasts treated with dASFC-CM (%) at different concentrations of cytotoxicity in accordance with at least one embodiment of the present disclosure. NC: negative control.

FIG. 8 shows the relative viability (%) of NC (negative control), PC (positive control) and dAFSC-CM 10% of an in vitro skin irritation test in accordance with at least one embodiment of the present disclosure. PC: positive control; NC: negative control.

FIG. 9 shows mean tissue viability (%) of NC (negative control), PC (positive control), and dAFSC-CM 10% of an in vitro eye irritation test in accordance with at least one embodiment of the present disclosure.

FIG. 10 shows the relative cell growth (%) of human hair follicle dermal papilla cells (HFDPC) treated with dASFC-CM at different concentrations in accordance with at least one embodiment of the present disclosure. Ctrl: negative control.

FIG. 11 shows the effect of dAFSC-CM on cell migration of HFDPC. HFDPC were treated with different concentrations (1%, 2%, 5%, 10%, and 15%) of dAFSC-CM for different incubation times (0, 5, 9, and 24 hours) in accordance with at least one embodiment of the present disclosure. Ctrl: negative control.

### DETAILED DESCRIPTION

The following embodiments are provided to illustrate the present disclosure in detail. A person having ordinary skill in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

The articles “a”, “an”, and “the” are used herein to refer to one or to more than one (i.e., to at least one) of the grammatical object of the article. The term “or” is used interchangeably with the term “and/or” unless the context clearly indicates otherwise. The term “including” is used herein to mean, and is used interchangeably with, the phrase “including but not limited to.” The term “about” is used herein to mean within the typical ranges of tolerances in the art. For example, “about” can be understood as about 2 standard deviations from the mean. When about is present before a series of numbers or a range, it is understood that “about” can modify each of the numbers in the series or range. For example, the numerical value is meant to encompass variations of ±20%, ±10%, ±5%, ±1%, ±0.5%, or ±0.1% from the numerical value. The numeral ranges used herein are inclusive and combinable, any numeral value that falls within the numeral scope herein could be taken as a maximum or minimum value to derive the sub-ranges therefrom. For example, it should be understood that the numeral range “20-30%” comprises any sub-ranges between the minimum value of 20% to the maximum value of 30%, such as the sub-ranges from 20% to 25%, from 25% to 30%, and from 22.5% to 27.5%. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, the terms “effective amount,” refers to the amount of an active agent or a pharmaceutical composition that is sufficient to bring about a cosmetic or therapeutic effect on a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refers to the placement of an active ingredient into a subject by a method or route which results in at least partial localization of the active ingredient at a desired site to produce the desired effect. For example, the active ingredient of the present disclosure may be administered to the subject by injection or topical administration, but the present disclosure is not limited thereto.

As used herein, “subject” is used to mean any vertebrate including, but not limited to, humans or mammals such as deer, mule, elk, mule deer. In some preferable embodiments, the subject is a mammal such as a humanor a non-human mammal, such as a domesticated mammal, e.g., a dog, a cat, a horse, a rat, a mouse, or the like, or a production mammal, e.g., a cow, a sheep, a pig, a deer, or the like.

The terms “comprise,” “comprising,” “include,” “including,” “have,” “having,” “contain,” “containing,” and any other variations thereof are intended used herein to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the terms “effective amount,” refers to the amount of an active agent or a pharmaceutical composition that is sufficient to bring about a cosmetic or therapeutic effect on a subject in need thereof. For example, the “cosmetically effective amount” may be the quantity of a composition provided for administration and at a particular dosing regimen that is sufficient to achieve a desired appearance, feel, and or protective effect. In some embodiment, the effective amount of the composition results in the prevention of or a decrease in the appearance and or symptoms associated with an undesirable condition, such as wrinkles, fine lines, skin thinness, loss of skin elasticity or suppleness, or other characteristics of skin associated with aging, UV, chemical exposure, adverse climate (e.g., temperature, humidity), dietary intake, biological agents, environmental oxidants, among others. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refers to the placement of an active ingredient into a subject by a method or route which results in at least partial localization of the active ingredient at a desired site to produce the desired effect. For example, the active ingredient of the present disclosure may be administered to the subject by injection or topical administration, but the present disclosure is not limited thereto. Administration of the compositions of the present disclosure may be performed systemically, or in a localized environment in the subject. For example, a site of local administration can be any site in the body in which the development of tissue is desired or beneficial, such as a joint, a surgical site, a site of a segmented skeletal gap or non-union fracture, a wound, an ulcer, or an inflammatory skin rash. Further, compositions of the present disclosure may be administered to a subject in, on or as part of an implantable device. For example, such a device can be a sponge, biocompatible polymer, bioerodible polymer, putty, gel, bone matrix, artificial bone matrix, bolt, screw, endotracheal tube, stent, contact lens, pacemaker, central IV tube, foley catheter, or intracranial device.

As used herein, “skin” as an enclosing organ, is used to mean the body's largest and fastest-growing organ, and is classified as the primary component of the integumentary system, one of the ten macro-organ systems found in “advanced” animals. Skin is particularly susceptible to disease and trauma due to its protective function. Skin fulfills several critical roles including regulating temperature, providing a dynamic barrier to the external world, repair processes occur in it throughout life and serving as a conduit to support an immense network of sensory receptors. The skin performs several functions that are vital to the survival and health of the body. The skin heals to prevent the loss of blood after wounds, regulates body temperature by dissipating heat and as a layer against cold, absorption, secretion, thermal-regulation, sensory detection and orientation, and barrier protection.

As used herein, “age-related features” or “wrinkle” is used to mean a slight line, small furrow, creases, folds, or ridges, or fold in especially the skin of the face, neck, arm, or any part of the body parts.

As used herein, “derivatives” of cells include, but not limited to, multipotent stem cells, pluripotent stem cells, adult stem cells, tissue specific stem cells, a secretome, a conditioned medium, an exosome, an extracellular-vehicle, a secreted substance, and any combination thereof.

As used herein, the term “cosmetically or pharmaceutically acceptable carrier” refers to a cosmetically or pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexation agent, wetting agent, or any combination thereof. In some embodiments, each component is “cosmetically or pharmaceutically acceptable” in the sense of being compatible with the other ingredients of a cosmetic or pharmaceutical formulation, and suitable for use in contact with the organ or tissue of a subject (e.g., a mammal) without excessive toxicity, allergic response, irritation, immunogenicity, or other complications or problems. Sec, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. The “carrier” used herein may be solid, solutions, or mixtures in which stem cells and or their derivatives are suspended for use in the protocol for topical treatment, transplantation or for any other subsequent uses. Such carriers include, but not limited to, gels, ointments, pastes, or aerosol sprays. In some embodiment, “cosmetically acceptable vehicle” refers to a medium that is compatible with keratin materials such as human skin.

As used herein, “wound” may be a site of the subject's outer body to be treated. The wound may be a site of a severe injury such as a burn, traumatic acute, chronic or surgical wound for which the site has a compromised and/or missing outer layer of skin. Examples of wounds may include both open and closed wounds. Wounds include, for example, burns, incisions, excisions, lacerations, abrasions, puncture or penetrating wounds, surgical wounds, contusions, hematomas, crushing injuries, and ulcers, but the present disclosure is not limited thereto.

### Wounds and Wound Classification

In addition to the definition previously provided, the term “wound” may also include for example, injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds. The methods of the present invention contemplate treating all wound types, including deep wounds and chronic wounds. The term “chronic wound” refers to a wound that has not healed. Preferably, it is selected from the group consisting of venous ulcers, pressure sores, vasculitic ulcers, diabetic ulcers and decubitus ulcers. Chronic skin wounds include, for example, pressure ulcers, diabetic ulcers, venous ulcers, vasculitic ulcers, arterial ulcers, and mixed ulcers. The chronic wound may be an arterial ulcer which comprises ulcerations resulting from complete or partial arterial blockage. The chronic wound may be a venous stasis ulcer which comprises ulcerations resulting from a malfunction of the venous valve and the associated vascular disease. The chronic wound may be a trauma-induced ulcer, a diabetic ulcer, or a vasculitic ulcer.

As used herein, “administer,” “administration,” “treatment,” “supplementation,” “injection,” or “provide” refers to a technique used to deliver a substance, i.e., stem cells into the body systemically or locally, or any combination thereof. When administering a therapeutically effective amount of the present invention parenterally or intravenously, it is generally formulated in a unit dosage form (e.g., emulsion, pills, and ointment).

It has been found that the supplementation/treatment with stem cells into the subject in need thereof, they can migrate/circulate into the target area, where the stem cells differentiate into adipocytes, hair follicle cells, skin cells, osteocytes, and chondrocytes and form structures including fat, hair, skin, and bone, restoring the functions in various subjects.

As used herein, “stem cells” may encompass cells derived from amniotic fluid, bone marrow, umbilical cord blood, umbilical cord, placental tissue, adipose tissue, peripheral blood, dental pulp and antler, but the present disclosure is not limited thereto. The stem cells can self-proliferate and differentiate into at least two lineages of cells. Further, “stem cell” as used herein may also refer to a cell that has the ability to replicate for indefinite periods, often throughout the life of an organism, and which, under certain conditions or given particular signals, can differentiate into many different cell types that make up the organism. That is, stem cells have the potential to develop into mature cells that have characteristic shapes and specialized functions, such as keratinocytes, sebocytes, transit amplifying cells, or melanocytes. Stem cells may reside in the epithelial basal layer, interfollicular epidermis (IFE) niches, sebaceous gland and hair follicle bulge.

As used herein, “cell” refers to the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and cukaryotes. The cells contemplated for use in this invention include all types of cells from all organisms in all kingdoms: plans, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

As used herein, the term “differentiation” related to cells means the process by which cells become structurally and functionally specialized, which is a progressive restriction of the developmental potential and increasing specialization of function which takes place during the development of the embryo and leads to the formation of specialized cells, tissues, and organs. The term “differentiable” means the ability of a cell to differentiate into a desired cell type. As used herein, the term “differentiates” means specialization (differentiation) or return to a more primitive cell type (dedifferentiation).

As used herein, “medium,” “basal medium,” or “media” refers to an optimal culture medium for the cultivation of a variety of animal cells, including neurons, stem cells, mesenchymal stem cells, primary epithelial cells, keratinocytes, cervical epithelial cells, kidney epithelial cells, and established cell lines, but not limited thereto. In at least one embodiment, the cultivation may be expansion or differentiation.

“Growth media” are composition used to grow microorganisms or cells in culture. There are different sorts of media for growing different sorts of cells. The biggest differences in growth media are between those used for growing cells in culture (cell culture uses specific cell types derived from plants or animals) and those used for growing microorganisms. These differences arise due to the fact that cells derived from whole organisms and grown in culture are often incapable of growth without the provision of certain requirements, such as hormones or growth factors which usually occur in vivo. In the case of animal cells these requirements are often provided by the addition of blood serum to the medium. These media are often red or pink due to the inclusion of pHI indicators. Growth media for embryonic stem cells preferably contains minimal essential medium, i.e., Eagle's: amino acids, salts (Ferric nitrate nonahydrate, Potassium chloride, Magnesium sulfate, Sodium chloride, Sodium dihydrogen phosphate), vitamins, (Ascorbic acid, Folic acid, Nicotinamide, Riboflavin, B-12) or Dulbecco's: additionally iron, glucose; non-essential amino acids, sodium pyruvate, β-mercaptocthanol, L-glutamine, fetal bovine serum and Leukemia Inhibitory Factor (LIF). In the case of microorganisms, there are no such limitations as they are often single cell organisms. One other major difference is that animal cells in culture are often grown on a flat surface to which they attach, and the medium is provided in a liquid form, which covers the cells. Bacteria such as Escherichia coli (E. coli, the most commonly used microbe in laboratories) may be grown on solid media or in liquid media, liquid nutrient medium is commonly called nutrient broth. The preferred growth media for microorganisms are nutrient broth or Luria-Bertani medium (L-B medium). Bacteria grown in liquid cultures often form colloidal suspensions. When agar (a substance which sets into a gel) is added to a liquid medium it can be poured into Petri dishes where it will solidify (these are called agar plates) and provide a solid medium on which microbes may be cultured.

One of skill in the art will be able to determine a suitable growth medium for initial preparation of stem cells. Commonly used growth media for stem cells includes, but is not limited to, Iscove's modified Dulbecco's Media (IMDM) media, DMEM, KO-DMEM, DMEM/F12, RPMI 1640 medium, McCoy's 5A medium, minimum essential medium alpha medium (α-MEM), F-12K nutrient mixture medium (Kaighn's modification, F-12K), X-vivo 20, Stemline, CC100, H2000, Stemspan, MCDB 131 Medium, Basal Media Eagle (BME), Glasgow Minimum Essential Media, Modified Eagle Medium (MEM), Opti-MEM I Reduced Serum Media, Waymouth's MB 752/1 Media, Williams Media E, Medium NCTC-109, neuroplasma medium, BGJb Medium, Brinster's BMOC-3 Medium, CMRL Medium, CO2-Independent Medium, Leibovitz's L-15 Media, and the like.

In at least one embodiment of the present disclosure, composition or cosmetic composition comprising: an effective amount of derived matrix produced from stem cell from amniotic fluid, wherein the amniotic fluid is obtained from a deer.

The subject of the present disclosure is a human, deer, a dog, a cat, a horse, a rat, a mouse, or the like, or a production mammal, e.g., a cow, a sheep, a pig.

The gestation period of the deer is between 4, 5, 6, 7, 8 or 9 months.

The composition or cosmetic composition produced from the amniotic fluid obtained from the deer exhibits properties that influence the regeneration of skin and its bio renewal which makes it possible to reconstruct age-related deterioration.

Furthermore, the composition or cosmetic composition produced from the amniotic fluid obtained from the deer exhibits properties that are useful as agents for the treatment of difficult to heal wounds arising, for example, as a result of diabetes, and skin damage following chemotherapy.

The cosmetic composition of the present disclosure is further useful in aesthetic medicine, particularly as agents for stimulating skin regeneration following burns, acne, cuts, and laceration due to physical trauma.

As used herein, the term “NANOG” refers to a homeobox gene. NANOG is thought to be required for stem cells to multiply without limit while remaining able to make different types of cells. The gene is potential master gene that helps make embryonic stem cells grow in the laboratory, making stem cells immortal.

As used herein, the term “SOX2” refers to the sex determining region Y (SRY) box 2 protein coding gene. This intronless gene encodes a member of the SRY-related HIMG-box (SOX) family of transcription factors involved in the regulation of embryonic development and in the determination of cell fate.

In at least one embodiment of the present disclosure, wrinkle is caused by age, obesity, injury, hormonal insufficiencies, drug side effects, or any combination thereof, but the present disclosure is not limited thereto. Moreover, wrinkle may naturally appear on skin as people get older.

In at least one embodiment of the present disclosure, the wound is caused by cut, burn, traumatic acute, chronic or surgical wound, or other impact. In addition, numerous causes may also lead to skin wounds such as lacerations, abrasion injuries, scalds, diabetic ulcers and bedsores caused by long-term bed rest. Wound healing is a complex and slow process in which new epithelial tissue and connective tissue are formed by the migration and proliferation during treatment.

In at least one embodiment of the present disclosure, the stem cells may be derived from amniotic fluid, bone marrow, umbilical cord blood, umbilical cord, placental tissue, adipose tissue, peripheral blood, and dental pulp, but the present disclosure is not limited thereto. In at least one embodiment of the present disclosure, stem cells are from deer and cultured for 4-14 days for expansion. In some embodiments, the deer stem cells from amniotic fluid are positive for CD9, CD90, CD29, CD44, CD73, CD105, Nestin, Sox2 and NANOG and negative for CD31, CD34, and CD45 using RT-PCR and/or flow cytometry.

In the present disclosure, deer stem cells for skincare and hair promotion may be used alone or in combination with gauze, mask, ointment, or any combination thereof.

### EXAMPLES

Exemplary embodiments of the present disclosure are further described in the following examples, which should not be construed to limit the scope of the present disclosure.

### Materials and Methods

### Cell Culture

The amniotic fluid was collected from a 5-month pregnancy of farmed deer, provided by Taiwan Deer Velvet Biotechnologies (Tainan, Taiwan). The procedure was the same as amniocentesis, 20 ml of amniotic fluid was drawn by syringe with a long needle under the ultrasound guidance. Fresh amniotic fluid was filtered with 70 μm sterile cell strainer (Corning Falcon) and centrifuged 200×g, 5 min. The pellet was washed with phosphate-buffered saline and re-centrifuged. The cells were cultured in Minimum Essential medium Eagle, alpha modification (α-MEM) with 10% fetal bovine serum (FBS) and 100 U/ml penicillin, 100 μg/ml streptomycin (P/S) in a T75 flask and kept in 37° C., 5% CO2 and humidified atmosphere. The culture medium was changed twice each week. The nonadherent cells were removed by medium change. Cell colonies appeared after 5-7 days culture and reached 80-90% confluency during 10-14 days. Cells were dissociated with trypsin/EDTA solution and passed for expansion or preservation.

### RT-PCR

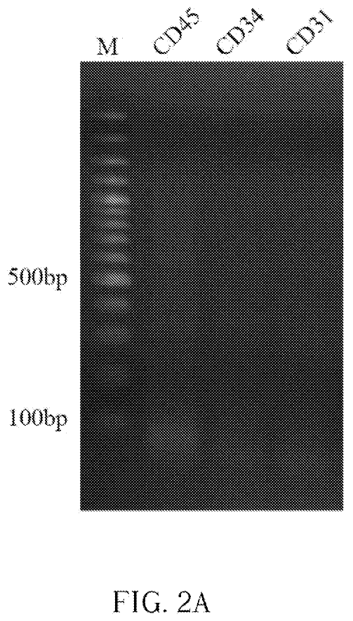

Total RNA was extracted from deer amniotic fluid stem cells using RNeasy® Mini kit (Qiagen) following the manufacturer's protocol. cDNA was synthesized from RNA using QuantiNova Reverse Transcription kit (Qiagen) and PCR amplifications by DreamTaq Green PCR master mix (Thermo Scientific). PCR reaction conditions were set for 2 min at 95° C., followed by 38 amplification cycles (95° C. for 30 s, 30 s at specific primer annealing temperature and 72° C. for 30 s and 72° C. for 15 min for final extension). All primers (listed in Table 1 (SEQ ID NO: 1˜20) or Table 2 (SEQ ID NO: 21˜26)) were designed by NCBI primers-pick and, the RT-PCR results were shown in FIG. 2A and FIG. 2B.

TABLE 1
The primers for CD9, CD29, CD44, CD73, CD90, CD105, Nestin, SOX2, NANOG,
and GAPDH were detected in this study by using RT-PCR.

| | Product size | | |
|---|---|---|---|
| Gene | (bp) | Gene number | Primer sequence (5′→3′) |
| CD9 | 435 | XM_043881342.1 | F: TTTGACCGGGGTGGTAGAAC |
| | | | R: TGCGGATGGCACAACATAGA |
| CD29 | 660 | XM_043882898.1 | F: CGGTAGTGCCTGTGACTGTT |
| | | | R: TCACCGGCAATTTAGAGACCA |
| CD44 | 413 | XM_043909244.1 | F: CCATCTGTGCTGCGAACAAC |
| | | | R: ATCGTTCCTATTGGTATCCGATGT |
| CD73 | 666 | XM_043889848.1 | F: AGCAGCATCCCAGAAGATCC |
| | | | R: TTTCCTCACAAAGTGGGAGCG |
| CD90 | 334 | XM_043901016.1 | F: CTGACAGTGGTGGTTCCAGTC |
| | | | R: TGTTGGAGACGGTGGGATTC |
| CD105 | 670 | XM_043916790.1 | F: CCTCCTCCAGGAAGTCCAAAAGAC |
| | | | R: CCTGGAGTTCTCACCTTCTGC |
| Nestin | 371 | XM_043876740.1 | F: TCCATCAGCGCTCCCATTTT |
| | | | R: GAGTGGGAGCGAGGCTATTC |
| Sox2 | 323 | XM_043874882.1 | F: CGCAAGATGGCCCAAGAGAA |
| | | | R: TTCATGTGCGCGTAGCTGTC |
| NANOG | 445 | XM_043881999.1 | F: CCATGTGGGGTAACCAGACC |
| | | | R: TCCTAAGGGGAAGCGGTACA |
| GAPDH | 536 | XM_043881239.1 | F: CCCTGGCCAAGGTCATTCAT |
| | | | R: ACCTTACTCCTTGGAGGCCA |

TABLE 2
The primers for CD31, CD34, and CD45 were not detected in this study
by using RT-PCR.

| Gene | Product size (bp) | Gene number | Primer sequence (5′→3′) |
|---|---|---|---|
| CD31 | 215 | XM_043905046.1 | F: TCGTCAGAAAAGGAGAGGGGA |
| | | | R: TTGGCGGTTAGTTCTGCGTT |
| CD34 | 332 | XM_043923458.1 | F: CACCCGTCACCTTAGTGTCT |
| | | | R: TAGCTTCAGGCAGATGCTGTG |
| CD45 | 326 | XM_043923195.1 | F: CCACCCAGGATGAGCAGAC |
| | | | R: ACTAGGATCTGGTGGTGCCT |

### Flow Cytometry Analysis

Surface markers for deer amniotic fluid stem cells were analyzed using flow cytometry by human-Nestin-FITC antibody (R&D System) and mouse/human-SOX2-APC antibody (R&D System). Isotype-matched mouse IgG1 FITC (R&D System) and mouse IgG2A APC (R&D System) were served as the negative controls.

### Differentiation Potential

To evaluate the tri-lineage differentiation potential of deer amniotic fluid stem cells, three different types of induction were conducted. Osteogenesis: The deer amniotic fluid cells were cultured on 6-well plate till 90% confluency. After PBS washing once, the medium was shifted to osteogenic medium, composed of DMEM with 10% FBS, 50 μM ascorbic acid 2-phosphate, 100 nM dexamethasone, 10 mM b-glycerophosphate (all Sigma-Aldrich). The medium was changed twice a week. After 3 weeks, Alizarin Red S staining was conducted to detect calcium deposition. Adipogenesis: The deer amniotic fluid cells were cultured on 6-well plate till 90% confluence. After PBS washing once, the medium was shifted to adipogenic medium, composed of DMEM with 10% FBS, 500μM 3-isobutyl-1-metyl-xanthine (IBMX), 1 μM dexamethasone, 60 μM indomethacin, and 5 μg/ml insulin (all Sigma-Aldrich). The medium was changed twice a week. After 3 weeks, Oil Red O staining was conducted to detect lipid droplet. Chondrogenesis: The deer amniotic fluid cells, 5×10⁵ cells were added in a 15-ml polypropylene conical centrifuge tube and centrifuged at 200×g, 5 min. Chondrogenic medium (MSCgo™ chondrogenic differentiation medium, Biological Industries cat. 05-220-1B) was added to the cell pellet and kept at 37° C. in a 5% CO2 incubator for 3 weeks. The medium was changed twice a week. After 4 weeks, the round pellets were embedded in paraffin and cut into 3-μm sections. The section on the slide was stained with Alcian blue to detect sulfated GAG as cartilage.

### Procollagen Synthesis

Human foreskin fibroblasts (Hs68) were seeded in 96-well plates and kept at 37° C. in a 5% CO2 incubator overnight. Culture medium was removed. Then, the fibroblasts were treated with dAFSC-CM (deer amniotic fluid stem cell conditioned medium) at different concentrations, culture medium (as negative control, NC) and 100 ng/ml TGF-β (as positive control, PC). Treated cells were kept at 37° C. in a 5% CO2 incubator for 72 hours. The supernatant was transferred to a microplate reader (Epoch™ Microplate Spectrophotometer) for measuring procollagen type I C-peptide levels. After measuring collagen in supernatant using microplate, the plate was added MTT solution and kept at 37° C. in a 5% CO2 incubator for 2 hours. MTT was used to form the purple Formazan crystal with the enzymes in mitochondria, thereby cell viability was determined. After reaction, DMSO was added to dissolve the crystal, and spectrophotometry was used at 570 nm wavelength for measuring OD value.

### A Measurement of Healing Area

Mouse fibroblasts (NIH/3T3) were seeded in 24-well plates containing SPLScar™ block. When the cells reached confluence, SPLScar™ block was removed. Then, the fibroblasts were treated with dAFSC-CM (deer amniotic fluid stem cell conditioned medium) at different concentrations, culture medium (NC) and 10 ng/ml TGF-β (positive control). Treated cells were kept at 37° C. in a 5% CO2 incubator for 8 hours. ImageJ was used to analyze the image of cell migration.

### A Measurement of Cell Viability

Mouse fibroblasts (NIH/3T3) were seeded in 96-well plates and kept at 37° C. in a 5% CO2 incubator overnight. Culture medium was removed. Then, the fibroblasts were treated with dAFSC-CM (deer amniotic fluid stem cell condition medium) at different concentrations and culture medium (NC). Treated cells were kept at 37° C. in a 5% CO2 incubator for 24 hours. The culture medium was change into MTT solution. The MTT was used to form the purple Formazan crystal with the enzymes in mitochondria, thereby cell viability was determined. After reaction, DMSO was added to dissolve the crystal, and spectrophotometry was used at 570 nm wavelength for measuring OD value.

### In Vitro Skin Irritation Test

dAFSC-CM (deer amniotic fluid stem cell conditioned medium) and 5% SDS (positive control) were added on top of EpiDerm™ tissue. All conditions were repeated in triplicate in accordance to EpiDerm™ skin irritation test standard protocol (OECD TG 439) for evaluating whether dAFSC-CM induced the skin irritation. After exposure to dAFSC-CM for 60 minutes, the tissue was washed with PBS and dried. Then, it was transferred to new culture medium and kept at 37° C. in a 5% CO2 incubator for 42 hours. The tissues were incubated with MTT solution (1 mg/mL) and kept at 37° C. in a 5% CO2 incubator for 3 hours for measuring cell relative viability (%). MTT was used to form the purple Formazan crystal with the enzymes in mitochondria, thereby cell viability was determined. After reaction, isopropanol was added to dissolve the crystals, and spectrophotometry was used at 570 nm wavelength for measuring OD value.

### In Vitro Eye Irritation Test

dAFSC-CM (deer amniotic fluid stem cell conditioned medium), sterile deionized water (NC), and methyl acetate (PC) were added on top of EpiOcular™ tissue. All conditions were repeated in duplicated in accordance to EpiOcular™ eye irritation test standard protocol (OECD TG 492) for evaluating whether dAFSC-CM induced the eye irritation. After exposure to dAFSC-CM for certain time, the tissue was washed with PBS and dried. Then, it was transferred to new culture medium and kept at 37° C. in a 5% CO2 incubator for re-incubation. The tissue was incubated with MTT solution (1 mg/mL) and kept at 37° C. in a 5% CO2 incubator for 3 hours for measuring cell relative viability (%). MTT was used to form the purple Formazan crystal with the enzymes in mitochondria, thereby cell viability was determined. After reaction, isopropanol was added to dissolve the crystal, and spectrophotometry was used at 570 nm wavelength for measuring OD value.

### Human Hair Follicle Dermal Papilla Cells (HFDPC) for Cell Proliferation and Migration Tests

For HFDPC proliferation test, HFDPC were seeded as 1,500 cells/well into 96-well plate and kept for cell adherance for 4 hours at 37° C. After washing twice with sterile PBS buffer, HFDPC were cultured in aMEM medium plus 2% FBS with different concentrations of dAFSC-CM (0%, 1%, 2%, 5%, 10%, and 15%) for 48 hours at 37° C. Cell growth was assessed by Cell Counting KIT-8 (Dojindo Laboratories, Gaithersburg, MD) following the manufacture's protocol and read the absorbance at 450 nm by microplate reader. All experiments were operated in triplicate and analyzed statistically by student t-test (p<0.05). For HFDPC migration assay, HFDPC cells were seeded into 24-well plate for 3 days at 37° C. till 90% confluency. After washing twice with sterile PBS buffer, HFDPC cells were artificially scratched by 200 μl yellow tip and re-cultured in aMEM medium plus 2% FBS with different concentrations of dAFSC-CM (0%, 1%, 2%, 5%, 10%, and 15%) for 0, 5, 9, 24 hours at 37° C. Cell migration images were captured at the beginning and at regular intervals and the closure arca was analyzed by ImageJ software.

### Example 1: Characterization of Deer Amniotic Fluid Stem Cells

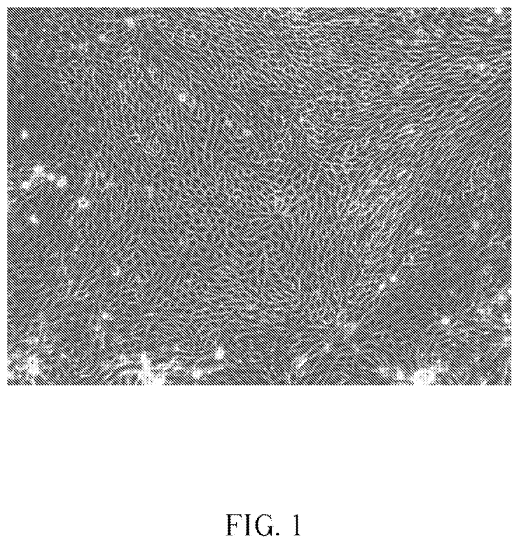

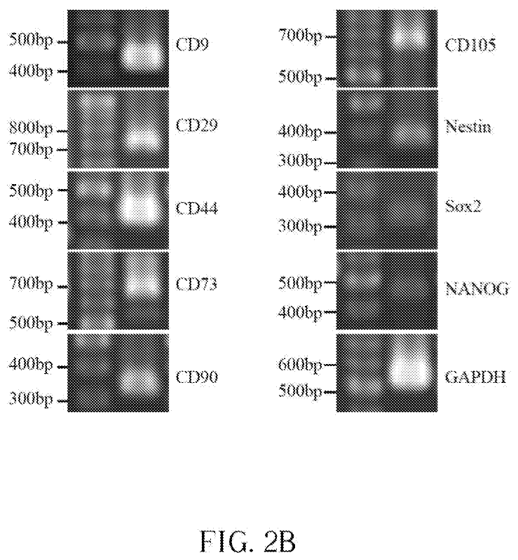

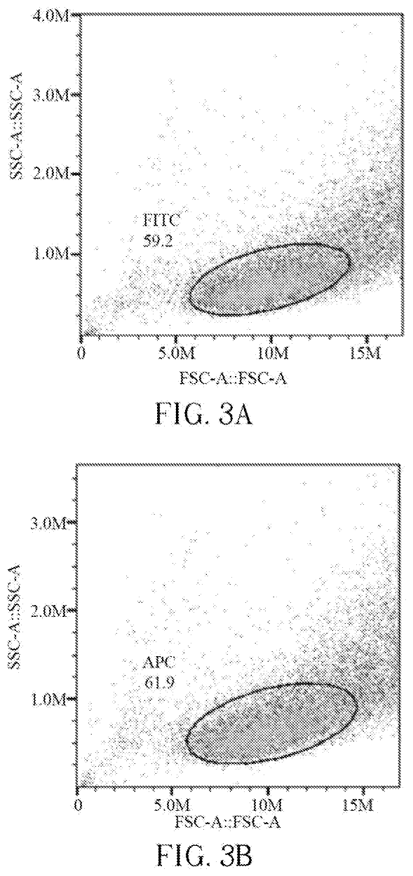

As shown in FIG. 1, the image under microscope revealed that cultured cells originated from deer amniotic fluid showed the fibroblast-like morphology, indicated deer amniotic fluid may contain stem cells. As shown in FIG. 2A, RT-PCT results showed that the cultured cells originated from deer amniotic fluid were negative for CD45 (lane 1), CD34 (lanes 2), and CD31 (lanes 3). As shown in FIG. 2B, RT-PCT results showed that the cultured cells originated from deer amniotic fluid were positive for CD9, CD29, CD44, CD73, CD90, CD105, Nestin, Sox2, and NANOG. The primer sequences for the above genes were listed in Tables 1 and 2. As shown in FIGS. 3A-3D, expressions of indicated antigen are shown in red histograms in contrast to isotype controls (black histograms). Values showed positive expression patterns (Nestin=98.5 and SOX2=92.5, respectively). As shown in FIG. 4A and FIG. 4B, under the osteogenic differentiation conditions, the cultured cells were strongly positively stained with Alizarin Red S (FIG. 4A: control, non-induction; 4B: after induction). As shown in FIG. 4C and FIG. 4D, the cultured cells in adipogenic differentiation media exhibited lipid droplets and showed positive staining with Oil Red O stain after 3 weeks of culture (FIG. 4C: control, non-induction; FIG. 4D: after induction). As shown in FIG. 4E and FIG. 4F, the cultured cells in chondrogenic differentiation media showed positive staining with Alcian Blue after 4 weeks of culture. These results indicated that the cultured cells are deer amniotic fluid stem cells.

### Example 2: In Vitro Functional Assay for Conditional Medium of Deer Amniotic Fluid Stem Cells (dASFC-CM)

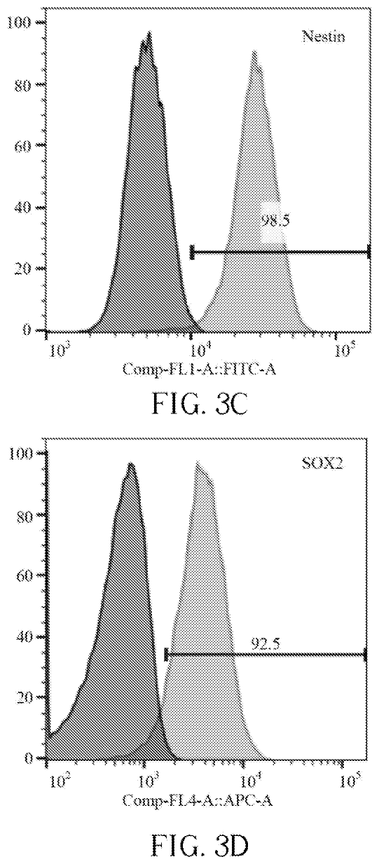

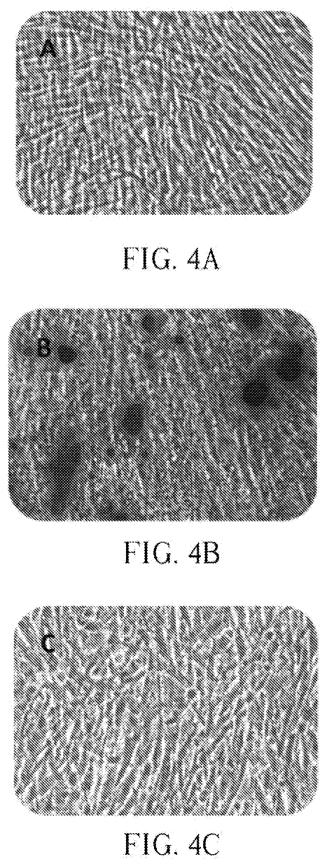

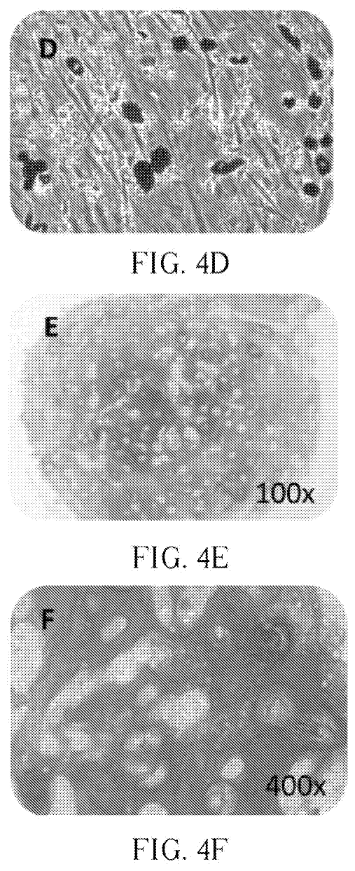

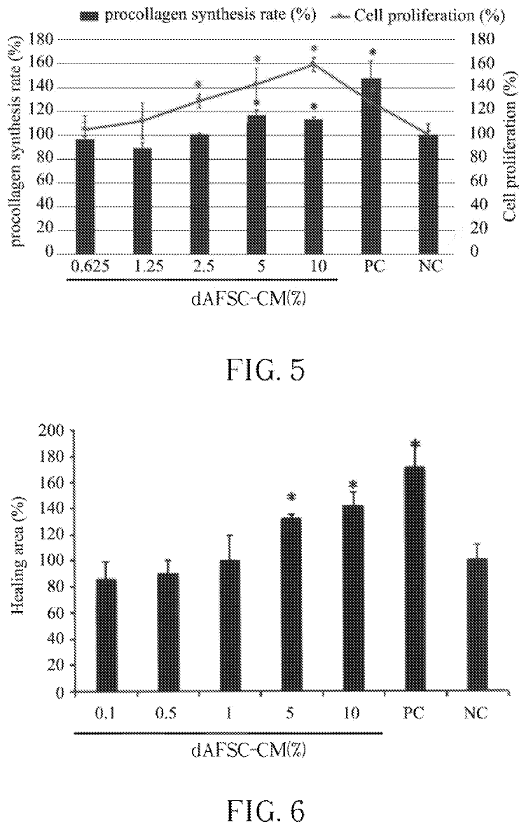

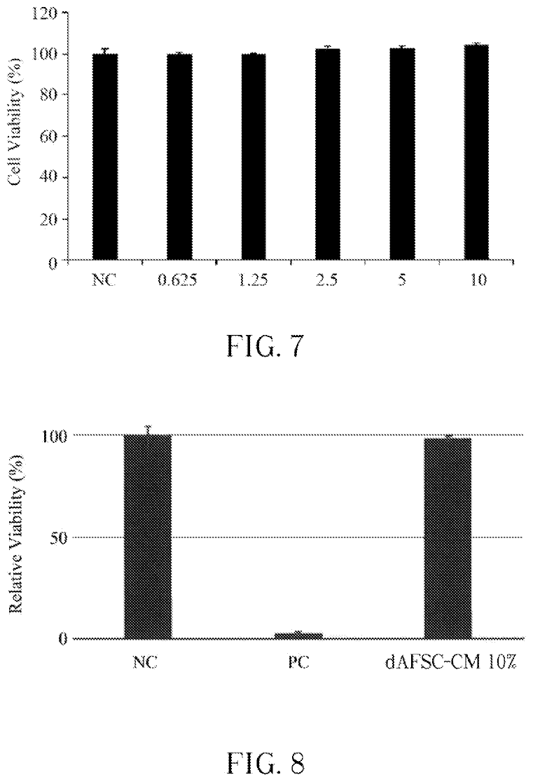

As shown in FIG. 5, dAFSC-CM at concentration of 2.5%, 5%, and 10% significantly promoted cell proliferation of human fibroblasts, and the human fibroblasts proliferation at 2.5%, 5%, and 10% dAFSC-CM were 130%, 142.7%, and 159.6%, respectively. Moreover, dAFSC-CM at 5% and 10% significantly promoted procollagen synthesis in human fibroblasts, and the procollagen synthesis rate for 5% and 10% dAFSC-CM were 116.6% and 113%, respectively. FIG. 6 shows dAFSC-CM at concentration of 5% and 10% significantly promoted wound healing, and the healing arca in 5% and 10% dAFSC-CM were 132% and 142%, respectively. FIG. 7 shows the cell viability of the fibroblasts treated with different concentrations (the unit of the horizontal axis is %) of dASFC-CM, in which the untreated cells (negative control, NC) was regarded as 100% and the cell viability test shows no significant changes under different concentrations of dAFSC-CM, indicating that the dAFSC-CM does not induce cytotoxicity in fibroblasts. As shown in FIG. 8 and FIG. 9, the relative viability and mean tissue viability were not affected by 10% dAFSC-CM, indicating dAFSC-CM does not induce skin and eye irritation.

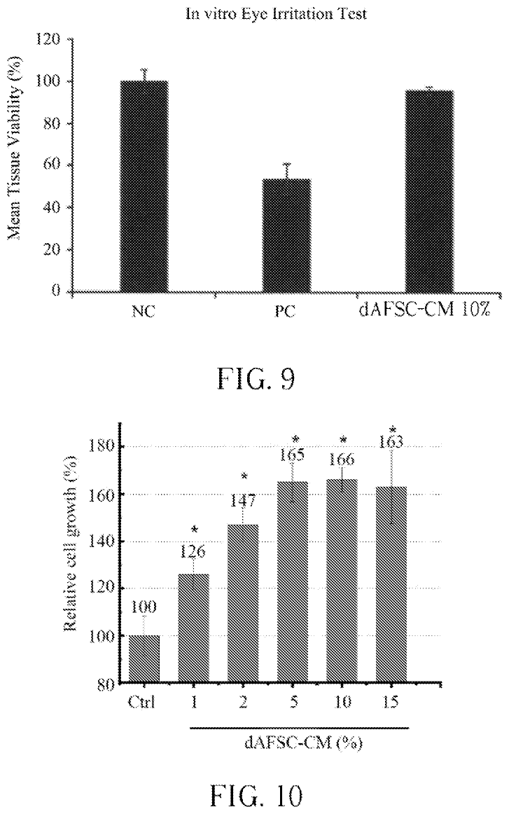

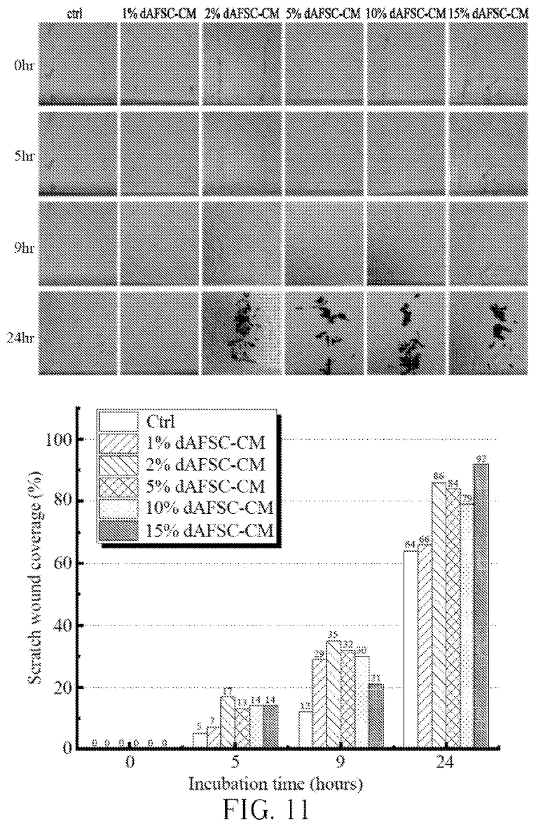

FIG. 10 shows the proliferation of human hair follicle dermal papilla cells (HFDPC) were stimulated significantly by dAFSC-CM at 1%, 2%, 5%, 10%, and 15% as compared with the control, and relative cell growth thereof were 126%, 147%, 165%, 166, and 163%, respectively. The data showed a saturated effect of above 5% of dAFSC-CM on the growth of HFDPC. FIG. 11 is the cell migration assay in which the recovery of scratch area was calculated by Image-J software comparison among images at 0, 5, 9, and 24 hours in different concentrations (1%, 2%, 5%, 10%, and 15%) of dAFSC-CM. The data showed the wound coverage was improved sufficiently by dAFSC-CM at 2%, 5%, 10%, and 15% after 5 hours of incubation as compared with the negative control. After 9 hours of incubation, 2% dAFSC-CM showed an improved performance in scratch wound coverage. In addition, the scratch wound coverage has reached 17% with 2% dAFSC-CM within 5 hours of incubation and the scratch wound coverage has reached at least 86% with 2% dAFSC-CM within 24 hours of incubation.

The above-described descriptions of the detailed embodiments are to illustrate the preferred implementation according to the present disclosure, and it is not to limit the scope of the present disclosure. Accordingly, all modifications and variations completed by those with ordinary skill in the art should fall within the scope of the present disclosure defined by the appended claims.

Those skilled in the art will readily observe that numerous modifications and alterations of the device and method may be made while retaining the teachings of the invention. Accordingly, the above disclosure should be construed as limited only by the metes and bounds of the appended claims.

1. A composition, comprising:
a stem cell or a derivative thereof; and
a cosmetically or pharmaceutically acceptable carrier,
wherein the stem cell is derived from amniotic fluid of an artiodactyl animal.

2. The composition of claim 1, wherein the artiodactyl animal is selected from the group consisting of a pig, a peccary, a hippopotamus, an antelope, a deer, a giraffe, a camel, a llama, an alpacas, a sheep, a goats, and a cattle.

3. The composition of claim 2, wherein the artiodactyl animal is deer.

4. The composition of claim 1, wherein the derivative is selected from the group consisting of a secretome, a conditioned medium, an exosome, an extracellular-vehicle, a secreted substance, and any combination thereof.

5. The composition of claim 1, wherein the stem cell is negative for CD31, CD34, CD45, or any combination thereof.

6. The composition of claim 1, wherein the stem cell is positive for CD9, CD29, CD44, CD73, CD90, CD105, Nestin, Sox2, NANOG, or any combination thereof.

7. A use of a composition for skin rejuvenation or skin treatment, comprising administering an effective amount of the composition to a subject in need thereof, wherein the composition comprises:
a stem cell or a derivative thereof; and
a cosmetically or pharmaceutically acceptable carrier, wherein the stem cell is derived from amniotic fluid of an artiodactyl animal.

8. The use of claim 7, wherein the artiodactyl animal is deer.

9. The use of claim 7, wherein the derivative is selected from the group consisting of a secretome, conditioned medium, an exosome, an extracellular-vehicle, a secreted substance, and any combination thereof.

10. The use of claim 7, wherein the composition promotes synthesis of procollagen, collagen, or elastin in the subject.

11. The use of claim 7, wherein the skin rejuvenation comprises preventing, treating, or reducing an age-related feature on skin of the subject.

12. The use of claim 11, wherein the age-related feature is wrinkles, age spots, rough skin textures, dullness of skin, fine lines, visible pores, loss of skin architecture, or any combination thereof.

13. The use of claim 7, wherein the skin treatment comprises restoring damage of skin of the subject, reducing scar of damaged skin of the subject, enhancing restoration of the skin of the subject after a cosmetic or dermatological procedure, or preventing or reducing a wrinkle on the skin of the subject.

14. The use of claim 13, wherein the damage is a wound caused by burn, traumatic acute, chronic, surgical wound, or other physical or chemical impact, and the wrinkle is caused by age, obesity, injury, hormonal insufficiencies, drug side effects, or any combination thereof.

15. A use of the composition for promoting hair growth or preventing hair loss, comprising administering an effective amount of the composition to a subject in need thereof, wherein the composition comprises:
a stem cell or a derivative thereof; and
a cosmetically or pharmaceutically acceptable carrier, wherein the stem cell is derived from amniotic fluid of an artiodactyl animal.

16. The use of claim 15, wherein the hair loss is caused by a disorder selected from the group consisting of androgenic alopecia, alopecia areta, and telogen effulvium.

17. The use of claim 15, wherein the artiodactyl animal is deer.

18. The use of claim 15, wherein the derivative is selected from the group consisting of a secretome, conditioned medium, an exosome, an extracellular-vehicle, a secreted substance, and any combination thereof.

19. The use of claim 15, wherein the composition promoting growth or migration of hair follicle dermal papilla cells in the subject.


SEQUENCE LISTING (26 sequences)

SEQ ID NO: 1 - DNA, 20 bp, synthetic construct
  source 1..20: mol_type = other DNA
tttgaccggggtggtagaac

SEQ ID NO: 2 - DNA, 20 bp, synthetic construct
  source 1..20: mol_type = other DNA
tgcggatggcacaacataga

SEQ ID NO: 3 - DNA, 20 bp, synthetic construct
  source 1..20: mol_type = other DNA
cggtagtgcctgtgactgtt

SEQ ID NO: 4 - DNA, 21 bp, synthetic construct
  source 1..21: mol_type = other DNA
tcaccggcaatttagagacca

SEQ ID NO: 5 - DNA, 20 bp, synthetic construct
  source 1..20: mol_type = other DNA
ccatctgtgctgcgaacaac

SEQ ID NO: 6 - DNA, 24 bp, synthetic construct
  source 1..24: mol_type = other DNA
atcgttcctattggtatccgatgt

SEQ ID NO: 7 - DNA, 20 bp, synthetic construct
  source 1..20: mol_type = other DNA
agcagcatcccagaagatcc

SEQ ID NO: 8 - DNA, 21 bp, synthetic construct
  source 1..21: mol_type = other DNA
tttcctcacaaagtgggagcg

SEQ ID NO: 9 - DNA, 21 bp, synthetic construct
  source 1..21: mol_type = other DNA
ctgacagtggtggttccagtc

SEQ ID NO: 10 - DNA, 20 bp, synthetic construct
  source 1..20: mol_type = other DNA
tgttggagacggtgggattc

SEQ ID NO: 11 - DNA, 24 bp, synthetic construct
  source 1..24: mol_type = other DNA
cctcctccaggaagtccaaaagac

SEQ ID NO: 12 - DNA, 21 bp, synthetic construct
  source 1..21: mol_type = other DNA
cctggagttctcaccttctgc

SEQ ID NO: 13 - DNA, 20 bp, synthetic construct
  source 1..20: mol_type = other DNA
tccatcagcgctcccatttt

SEQ ID NO: 14 - DNA, 20 bp, synthetic construct
  source 1..20: mol_type = other DNA
gagtgggagcgaggctattc

SEQ ID NO: 15 - DNA, 20 bp, synthetic construct
  source 1..20: mol_type = other DNA
cgcaagatggcccaagagaa

SEQ ID NO: 16 - DNA, 20 bp, synthetic construct
  source 1..20: mol_type = other DNA
ttcatgtgcgcgtagctgtc

SEQ ID NO: 17 - DNA, 20 bp, synthetic construct
  source 1..20: mol_type = other DNA
ccatgtggggtaaccagacc

SEQ ID NO: 18 - DNA, 20 bp, synthetic construct
  source 1..20: mol_type = other DNA
tcctaaggggaagcggtaca

SEQ ID NO: 19 - DNA, 20 bp, synthetic construct
  source 1..20: mol_type = other DNA
ccctggccaaggtcattcat

SEQ ID NO: 20 - DNA, 20 bp, synthetic construct
  source 1..20: mol_type = other DNA
accttactccttggaggcca

SEQ ID NO: 21 - DNA, 21 bp, synthetic construct
  source 1..21: mol_type = other DNA
tcgtcagaaaaggagagggga

SEQ ID NO: 22 - DNA, 20 bp, synthetic construct
  source 1..20: mol_type = other DNA
ttggcggttagttctgcgtt

SEQ ID NO: 23 - DNA, 20 bp, synthetic construct
  source 1..20: mol_type = other DNA
cacccgtcaccttagtgtct

SEQ ID NO: 24 - DNA, 21 bp, synthetic construct
  source 1..21: mol_type = other DNA
tagcttcaggcagatgctgtg

SEQ ID NO: 25 - DNA, 19 bp, synthetic construct
  source 1..19: mol_type = other DNA
ccacccaggatgagcagac

SEQ ID NO: 26 - DNA, 20 bp, synthetic construct
  source 1..20: mol_type = other DNA
actaggatctggtggtgcct